SEQUENCE LISTING (7 sequences)

SEQ ID NO: 1 - DNA, 35 bp, Artificial Sequence
  Oligo Extension
tgcataatcaccactaaaactgtaaagctaagtga

SEQ ID NO: 2 - DNA, 35 bp, Artificial Sequence
  Oligo Extension
gttacgaaacacgctctaagtctctaaactcgaat

SEQ ID NO: 3 - DNA, 5 bp, Artificial Sequence
  linker sequence
tgcat

SEQ ID NO: 4 - DNA, 5 bp, Artificial Sequence
  linker sequence
cgaat

SEQ ID NO: 5 - DNA, 30 bp, Artificial Sequence
  part of Oligo Extension
aatcaccactaaaactgtaaagctaagtga

SEQ ID NO: 6 - DNA, 30 bp, Artificial Sequence
  part of Oligo Extension
gttacgaaacacgctctaagtctctaaact

SEQ ID NO: 7 - DNA, 44 bp, Artificial Sequence
  Aptamer
accgggaggatagttcggtggctgttcagggtctcctcccggtg

(19) United States
(12) Patent Application Publication
(10) Pub. No.: US 2018/0171382 A1
(43) Pub. Date: Jun. 21, 2018

(54) BRANCHED PROXIMITY HYBRIDIZATION ASSAY

(71) Applicants: Albert-Ludwigs-Universität Freiburg, Freiburg (DE); Max-Planck-Gesellschaft Zur Förderung Der Wissenchaften E.V., München (DE)

(72) Inventors: Marco Cavallari, Freiburg (DE); Jianying Yang, Freiburg (DE); Michael Reth, Freiburg (DE)

(21) Appl. No.: 15/739,354

(22) PCT Filed: Jun. 27, 2016
(86) PCT No.: PCT/EP2016/064864

§ 371 Date: Dec. 22, 2017

(30) Foreign Application Priority Data
Jun. 26, 2015 (EP) 15174094.1

(51) Int. Cl. C12Q 1/682; G01N 33/68

(52) U.S. Cl. CPC C12Q 1/682; G01N 33/6845; C12Q 2525/161; C12Q 2525/197; C12N 2310/50; C12Q 2525/313; C12Q 2543/10; C12Q 2565/102; C12Q 2525/205

(57) ABSTRACT

The invention relates to a method for detecting the proximity of at least two biomolecules using branched DNA technology. The assay is called branched proximity hybridization assay.

### BACKGROUND

Proteins can interact with different kind of molecules. Such interactions are related to their function and are therefore an object of study in molecular biology. Protein-protein and Protein-nucleic acid (Protein-NA) interaction are of particular interest.

Protein-protein interactions play an important role in regulating the physiological functions in cells, such as gene expression, transport, signal transduction and cell cycle control. Identification of the interacting protein partners and the contact sites involved is important for the understanding of protein functionalities and also assists in providing novel approaches for the development of treatment and diagnostic methods and compositions.

There are various methods to investigate protein-protein interactions. A basic method to detect protein-protein interaction is co-immunoprecipitation (co-IP). The protein of interest is isolated with a specific antibody. Interaction partners are subsequently identified by Western blotting. While co-IP will give temporal resolution, the spatial resolution is limited to the compartment the interaction partners are isolated from.

Proximity ligation assay is a technology used for direct and spatiotemporal—below the diffraction limit of light—detection of proteins, protein interactions and modifications. Two primary antibodies from different species bind to a target antigen. Species-specific secondary antibodies (each with a unique short DNA strand), bind to the primary antibodies. When the secondary antibodies are in close proximity, the DNA strands can interact through the subsequent addition of two other circle-forming DNA oligonucleotides. After joining the two added oligonucleotides by enzymatic ligation, they are amplified via a rolling circle amplification. After the amplification reaction, fluorescently labeled complementary oligonucleotide probes visualize the product. The resulting high concentration of fluorescence in each single-molecule amplification product can be detected using a fluorescence microscope.

Other methods known in the state of the art include bimolecular fluorescence complementation (BiFC), affinity electrophoresis, phage-display pull-down assays, yeast two-hybrid screens or label transfer. Each of the approaches has its own advantages and disadvantages, especially with regard to sensitivity and specificity of the method. Therefore, there is a continuing need for effective and simple methods to detect protein-protein interactions as well as protein-nucleic acid interactions.

Disadvantages of the methods known in the state of the art are that these methods are not linear, hardly quantifiable and not high-throughput.

Branched DNA (bDNA) signal-amplification technology has been extensively used to detect and quantify specific nucleic acid sequences. bDNA can be applied to the detection of nucleic acid targets for which a sequence is known without the use of radioactive probes.

Various bDNA assays are known in the state of the art. These are signal amplification assays that are used to detect nucleic acid molecules. In this assay oligonucleotides (called capture extenders) are used to capture a target to a solid support. The target is labeled by binding a large number of target-specific oligonucleotides (label extenders). The label extender probes bind a bDNA Pre-amplifier, which in turn bind many amplifiers. The amplifier binds alkine phosphatase or other label probes (like fluorescent label probe). The result is a strong signal amplification.

Another commercial branched DNA method known in the state of the art is a quantification method for RNA transcription, the so called PrimeFlow™ assay. The PrimeFlow™ assay detects RNA transcripts by hybridization of two distinct Z-DNA Probes (gene-specific label extenders) to the target region and amplifies the binding signal using bDNA technology by sequential hybridization of preamplifier, amplifier and finally fluorescence-labeled probes. The usage of two distinct Z-DNA Probes here reduces the possible off-target rate, ensuring high specificity of the assay. A standard flow cytometer is used for the detection.

### DESCRIPTION OF THE INVENTION

There remains a need in the state of the art to provide highly sensitive methods for detection of biomolecule proximity. The present invention satisfies these and other needs in the state of the art.

In light of the prior art the technical problem underlying the present invention is to provide an alternative and improved method for the detection of biomolecule proximity.

This problem is solved by the features of the independent claims. Preferred embodiments of the present invention are provided by the dependent claims.

The invention therefore relates in a first preferred embodiment to a method for detecting the proximity of at least two target biomolecules comprising:
Providing at least two target biomolecules,
providing at least two Target Binding Reagents, each binding to at least one of said target biomolecules,
wherein at least one designed Oligo Extension, comprising a linker and a complementary sequence to a Z-DNA Probe or a complementary sequence to a Pre-amplifier, is attached to each Target Binding Reagent,
binding of at least two Target Binding Reagents with different Oligo Extensions to the at least two target biomolecules and
hybridization of two Z-DNA Probes, that are in vicinity, to Pre-amplifiers, or hybridization of the sequence complementary to a Pre-amplifier to Pre-amplifiers,
forming a branched DNA structure, wherein said branched DNA structure comprises Pre-amplifiers and Amplifiers
hybridization of Label Probes and
detection.

The method of the invention detects the proximity of two target biomolecules using a branched DNA structure or branched DNA technology. It is therefore also called branched proximity hybridization assay (bPHA).

A branched DNA structure can also be called bDNA and can comprise Pre-amplifiers, Amplifiers and may comprise Label Probes. In the state of the art the bDNA technology is used to detect nucleic acids.

BDNA technology is achieved through a series of sequential hybridization steps. The result is a “tree”-like structure. Pre-amplifier molecules hybridize to their respective pair of bound Z-DNA Probes or Oligo Extensions to form the “trunk” of the tree. Multiple Amplifier molecules hybridize to their respective Pre-amplifier and therefore create the “branches.” In a next step, multiple Label Probes hybridize to the Amplifiers. A fully assembled “tree” can contain up to 400 Label Probe binding sites.

Both the Amplifier and the Pre-amplifier are ssDNA. Appropriate methods for synthesizing them are known to a skilled person and are not intended as limiting embodiments of the present invention. Also, modified bases can be used to improve the specificity.

The Pre-amplifier comprises at least two parts. The first part is complementary to a part of the two Z-DNA Probes or Oligo Extensions for detecting the two Z-DNA Probes/Oligo Extensions in vicinity. The second part of the Pre-amplifier contains more than 10, preferred more than 15, especially preferred 20 repeat sequences.

Similarly, the Amplifier comprises also two parts: one part is complementary to the repeat sequence of Pre-amplifier and the second part is composed of more than 10, preferred more than 15, especially preferred 20 repeat sequences for hybridizing with the Label Probes. Thus, a set of Pre-amplifier and Amplifier is able to amplify the signal up to 400 times.

It is a merit of the invention that this bDNA technology can now be used in another important field, namely in proximity assays or interaction assays. It was surprising that this technology can be adapted to a very different field without resulting in losses of multiplexing or linearity. The combination of elements of known assays resulted in a surprising effect, namely a multiplex ready proximity assay with a superior linearity.

The method of the invention is characterized inter alia by its high reproducibility.

It is preferred that a PrimeFlow™ assay is used in the method of the invention. Due to the Oligo Extensions and the Target Binding Reagents of the invention the PrimeFlow™ assay can be used to detect the proximity and/or interaction of two target biomolecules. The invention describes several original and novel designs which convert this state of the art RNA assay to a multiplex, high-throughput protein-protein proximity and/or protein-nuclear acid proximity assay.

It is especially that the eBioscience 88-18001 kit or parts thereof are used.

To detect biomolecule proximity, reagents binding to the target biomolecule(s) are needed. Two designed Oligo Extensions carrying each a specific Z-DNA Probe targeting sequence or a Pre-amplifier targeting sequence and a linker are attached to the Target Binding Reagents. If the target biomolecule(s) are in proximity, binding of the Target Binding Reagent(s) with these Oligo Extensions attached then bind the two Z-DNA Probe target sequences or a Pre-amplifier and allow the detection of the biomolecule interaction as for the verification of target RNA with the PrimeFlow™ assay. It is preferred that the signaling amplification step is identical to the current PrimeFlow™ assay. Therefore, this novel bPHA assay inherits the same multiplex and high-throughput features with a single cell resolution.

It is preferred that the linker is ssDNA. Especially preferred is the use of a linker with a size 3 to 15 nucleotides. Especially preferred are linkers with 5 nucleotides. Such linkers achieved satisfying results and did not interfere with the hybridization processes.

A Target Binding Reagent can be any (engineerable) structure that specifically binds another biomolecule.

In another preferred embodiment the invention relates to the method, wherein the Target Binding Reagent is a biomolecule, preferably selected from the group comprising nucleic acid sequences, aptamers, antibodies, fragment antigen-binding fragments (Fab), nanobodies, single-chain variable fragments (scFv), proteins, natural ligands of the target biomolecules and antibody-like proteins. Antibody-like protein are for example DARPins.

The at least two Target Binding Reagents can be different or identical biomolecules.

It is preferred that at least two different Oligo Extensions are used. It is especially preferred that the Oligo Extensions form pairs. Therefore, two Target Binding Reagents with different Oligo Extensions can bind to two biomolecules. Two biomolecules that are in vicinity are detected by a pair of Target Binding Reagents linked to Oligo Extensions. A Pre-amplifier can either hybridize directly with both Oligo Extensions or a Pre-amplifier can hybridize with both Z-DNA Probes that are bound to the Oligo Extensions.

Especially preferred are the following Oligo Extensions:

SEQ NO ID 1:
5′ TGCATAATCACCACTAAAACTGTAAAGCTAAGTGA 3′
SEQ NO ID 2:
5′ GTTACGAAACACGCTCTAAGTCTCTAAACTCGAAT 3′

Within these sequences the following sequences are linker sequences:

SEQ NO ID 3:
TGCAT (first 5 nucleotides of SEQ ID NO 1)
SEQ NO ID 4:
CGAAT (last 5 nucleotides of SEQ ID NO 2)

Within sequences ID NO 1 and 2 the following sequences are complementary to the Z-DNA Probe:

SEQ NO ID 5:
5′ AATCACCACTAAAACTGTAAAGCTAAGTGA 3′
SEQ NO ID 6:
5′ GTTACGAAACACGCTCTAAGTCTCTAAACT 3′

It is preferred that said two Z-DNA Probes bind to the complementary sequences of the Oligo Extension.

It is preferred that the Z-DNA Probe is a DNA molecule. It can contain modified bases or comprise a Polyethylene glycol (PEG) linker. It functions as a bridge between the Oligo Extensions linked to the Target Binding Reagents and the Pre-amplifier by hybridizing with both of them. It is preferred that the Z-DNA Probes form pairs that correspond to the Oligo Extension pairs. The Z-DNA Probe hybridizes with one part to an Oligo Extension and with another part to a Pre-amplifier. A pair of Z-DNA Probes hybridizes to the same Pre-amplifier, preferred in adjacent positions at the same time. Therefore one Pre-amplifier can detect the proximity of two Z-DNA Probes and therefore of two target biomolecules.

The advantage of using the Z-DNA Probe is, that it allows a modular design of the system and the method.

Especially suitable is the use of the Z-DNA Probes for the PrimeFlow™ system because the Pre-amplifier of this system can be used and no Pre-amplifiers have to be synthesized, which would be very costly. If the Z-DNA Probe is used, it is necessary to change the part of the Z-DNA Probe which will find the target RNA and keep the Pre-amplifier unchanged.

For the method of the invention, the use of Z-DNA Probes is also beneficial when it comes to multiplexing. It is possible to change the Z-DNA Probe in a simple manner and therefore change the system to another set of the bDNA structure.

It is also preferred that the method of the invention does not use Z-DNA Probes but that the Oligo Extensions comprise sequences that are directly complementary to a Pre-amplifier. In this case, published bDNA technology (other than the PrimeFlow™ approach) can be used for detection.

It is further preferred that the molar ratio between Target Binding Reagent and Oligo Extension is 1:1.

It is preferred that the method is a high-throughput method.

Also preferred is the method wherein the detection is a fluorescent or an enzymatic detection.

For a fluorescent detection, the proximity of the biomolecules can for example be detected, upon completion of the assay protocol, by analyzing the sample on a standard flow cytometer equipped with an appropriate laser and filter configuration to capture the fluorescent signals. Other appropriate methods are known to a skilled person and are not intended as limiting embodiments of the present invention.

Also preferred is that the Label Probes are fluorescently, biotin- or enzyme-labeled probes. As enzyme-labeled probes for example the alkaline phosphatase enzyme can be used. Appropriate labels are known to a skilled person and are not intended as limiting embodiments of the present invention. For example Alexa Fluor® 647, Alexa Fluor® 488 or Alexa Fluor® 750 can be used.

It is especially preferred that the method is multiplex ready.

In a preferred embodiment the target biomolecules are proteins or nucleic acids or modifications of those.

The term “protein”, as used herein, refers to a polymer of amino acid residues linked together by a peptide bond. The term is meant to include proteins and polypeptides of any size, structure, or function. Typically, however, a protein is at least 10 amino acids long. A protein may be naturally occurring, recombinant, or synthetic, or any combination thereof. A protein may also be a fragment of a naturally occurring protein. A protein may be a single molecule or may be a multi-molecular complex. The term protein may also apply to amino acid polymers in which one or more amino acid residues are artificial chemical analogues of the corresponding naturally occurring amino acid. An amino acid polymer in which one or more amino acid residues are “unnatural” amino acids not corresponding to any naturally occurring amino acid is also encompassed by the use of the term “protein” herein.

As used herein, “nucleic acid” shall mean any nucleic acid molecule including and without limitation DNA, RNA, and hybrids or modified variants thereof.

The at least two target biomolecules can be different or identical biomolecules.

It is especially preferred that the method is a protein-protein and/or a protein-nucleic acid proximity assay.

Protein-NA proximity assays can be realized by using one Oligo Extension-coupled Target Protein Binding Reagent and one Oligo Extension that carries both the Z-DNA Probe target sequence or the Pre-amplifier target sequence and the sequence complementary to the target DNA or RNA sequence. This means that in this embodiment the Oligo Extension acts as a Target Binding Reagent. With this preferred approach it is possible to detect the proximity of the target protein and the target nucleic acids.

It is also preferred that the method is linear over a broad range, preferred at least two logarithmic scales.

In another preferred embodiment of the invention the designed Oligo Extension is coupled to a Target Binding Reagent via
(i) Chemical crosslinking
(ii) Labeling the Target Binding Reagent with a designed Oligo Extension using a sortase transpeptidation reaction, or
(iii) Nucleic acid synthesis.

It is preferred that Sulfo-SMCC and a Thio-modified oligonucleotides are used for the chemical crosslinking. Target Binding Reagents selected from the group comprising target binding antibodies, Fab fragments, nanobodies and scFv are particularly suitable for the chemical crosslinking method.

It was surprising that it is possible to specifically label target specific scFv or nanobody with only one oligonucleotide to achieve a complete linear amplification of the proximity detection, allowing more precise quantification. For that, a preferred embodiment of the invention takes advantage of the sortase transpeptidase. Target specific scFv and nanobodies are produced in E. coli with a (usually c-terminal) LPXTG amino acid motif. N-terminally free GGG peptide is chemically synthesized to the Oligo Extensions. Then, the GGG-coupled Oligo Extensions are attached to the nanobodies or scFv by means of their LPXTG motif through a sortase mediated transpepdidation reaction.

A preferred 1:1 Oligo Extension/Target Binding Reagent ratio can also be achieved with the aptamer technology. A DNA aptamer against a target biomolecule is developed in vitro by the SELEX or another suitable technique. The Z-DNA Probe target sequence or Pre-amplifier target sequence can then be easily attached to the aptamer sequence simply through nucleic acid synthesis.

In another preferred embodiment the invention relates to a kit for the use in a described method, comprising Target Binding Reagents with attached and designed Oligo Extensions, Pre-amplifiers, Amplifiers and Label Probes.

It is preferred that the kit further comprises Z-DNA Probes.

In another preferred embodiment the invention related to the use of a described method or a described kit in medical diagnostics. Especially preferred is the use of the method or the kit for point-of-care diagnostics.

### FIGURES

The invention is further described by the figures. These are not intended to limit the scope of the invention.

Short description of the figures:

FIG. 1: Design of a preferred protein-protein proximity assay of the invention.

FIG. 2: Design of a preferred protein-nucleic acid proximity assay of the invention.

FIG. 3: Coupling through chemical crosslinking

FIG. 4: Coupling using sortase transpeptidase

FIG. 5: Coupling using aptamers

FIG. 6: Protein-protein proximity: Detection of BCR oligomers (aptamer)

FIG. 7: Protein-protein proximity: Detection of membrane associated immunoglobulin heavy chain dimers (nanobody)

FIG. 8: Protein-protein proximity: Detection of cytokine receptor homodimerization on the cell surface (antibody)

FIG. 9: Results of bPHA experiments

FIG. 10: Linearity comparison

FIG. 11: Data collection and speed comparison

FIG. 12: Design of a preferred protein-protein proximity assay of the invention.

FIG. 13: Ramos BCR-specific TD05 aptamer design

### DETAILED DESCRIPTION OF THE FIGURES

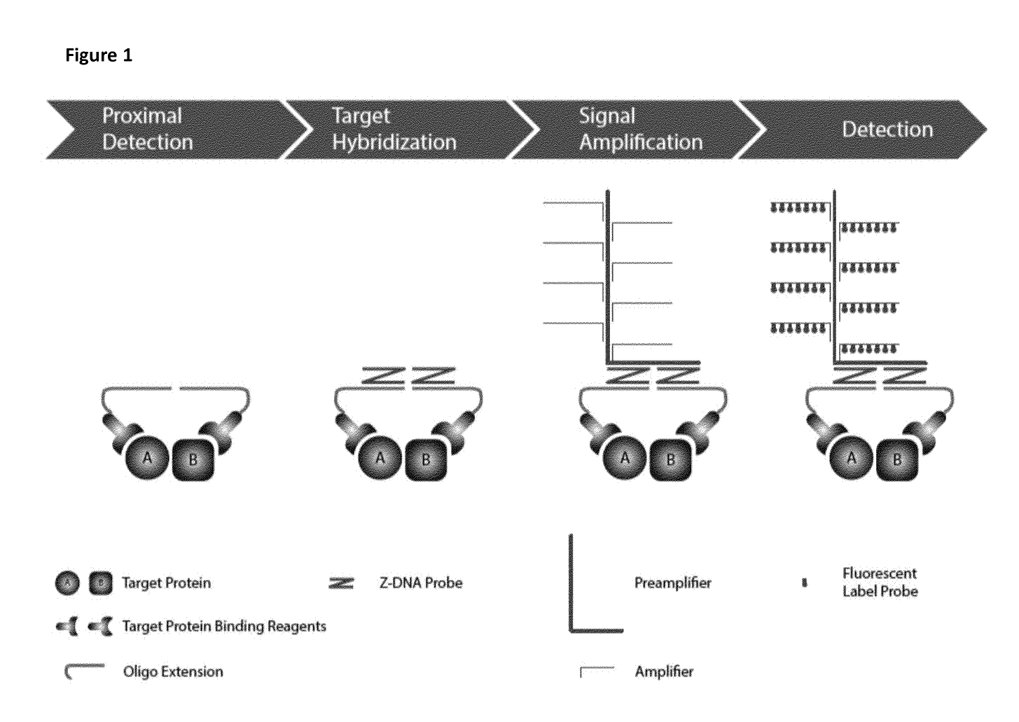

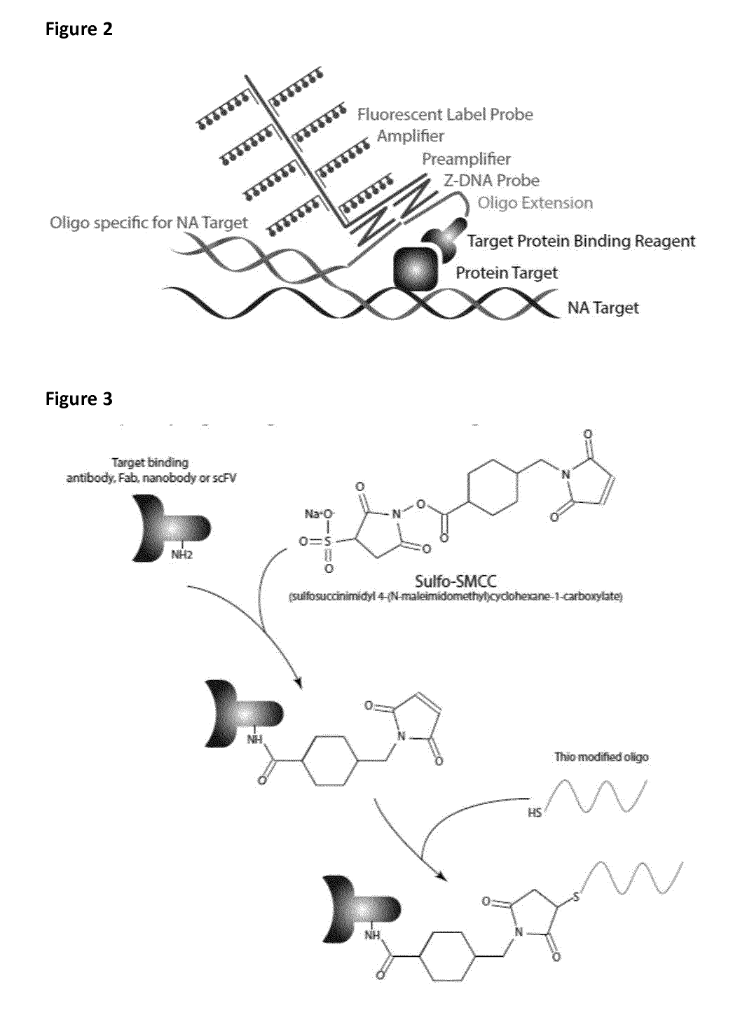

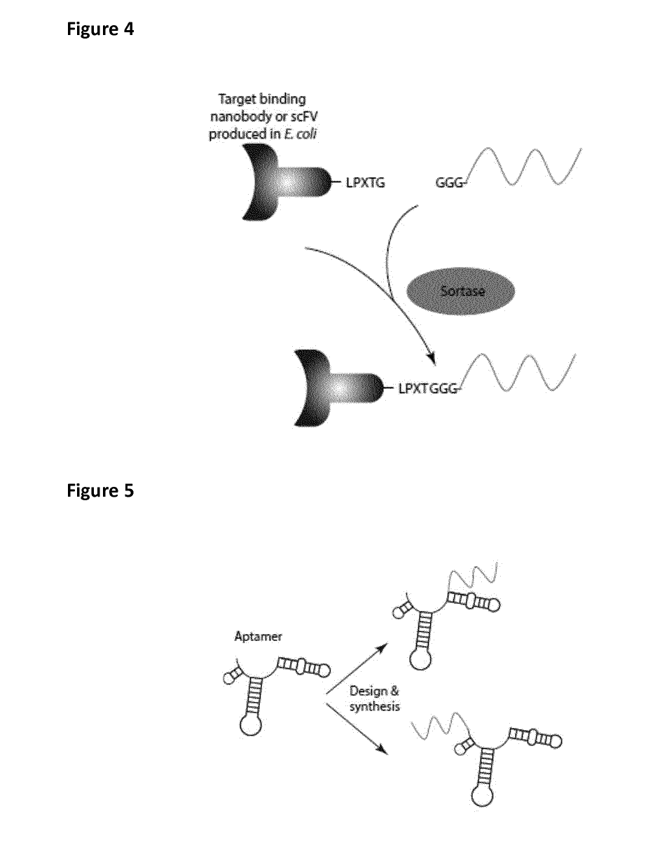

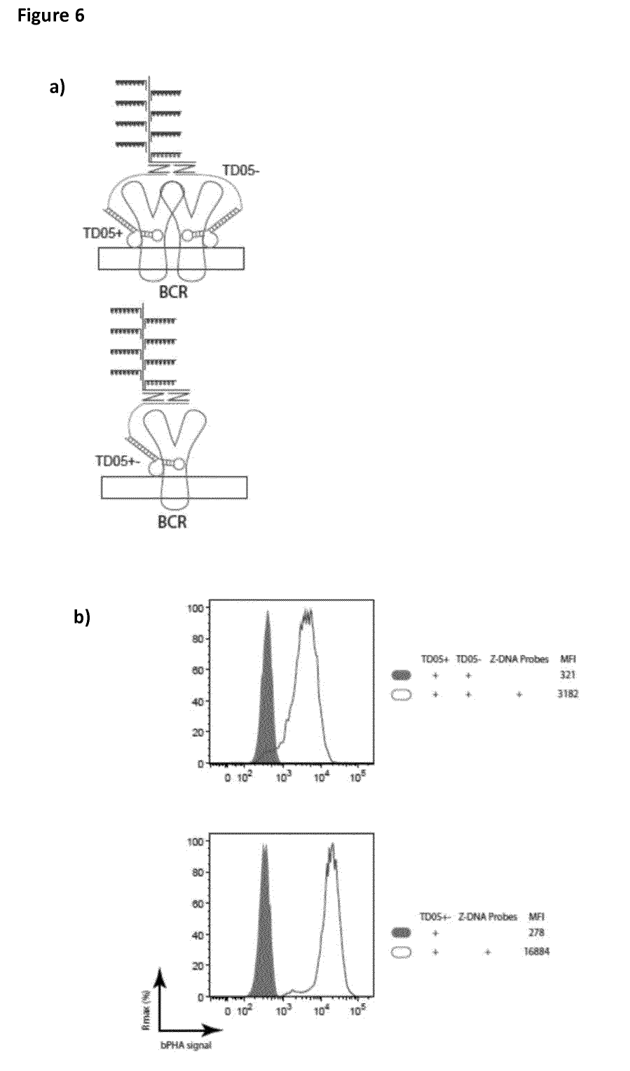

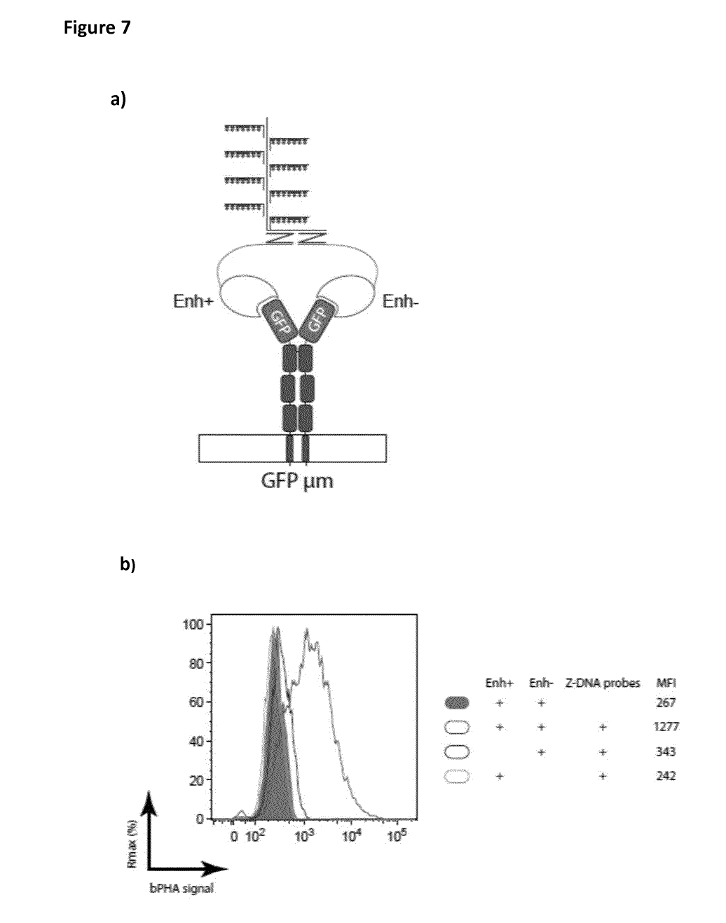

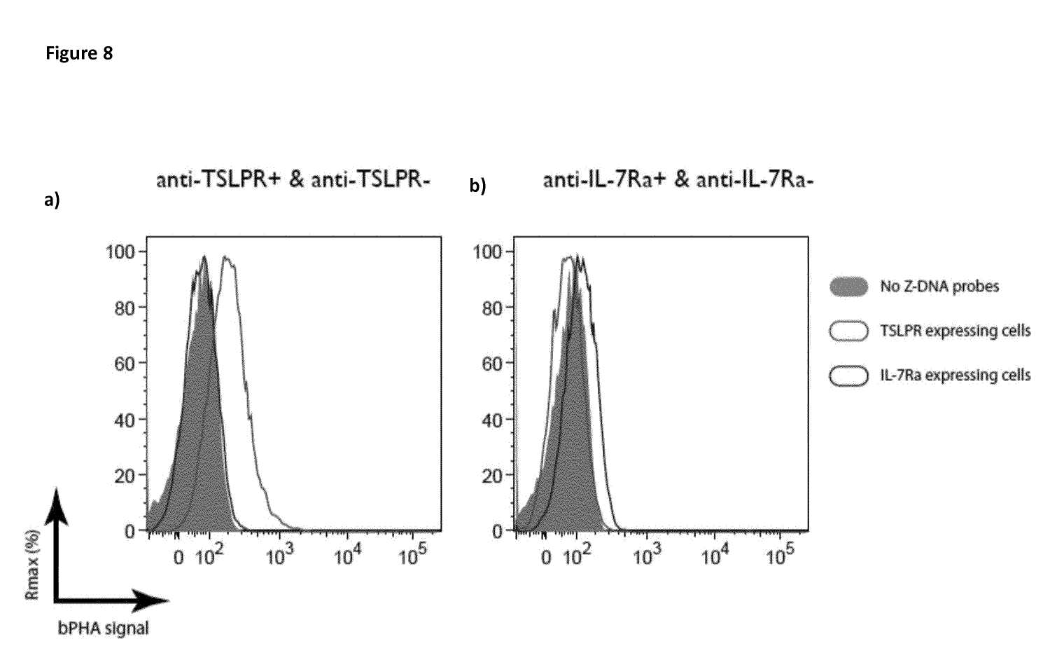

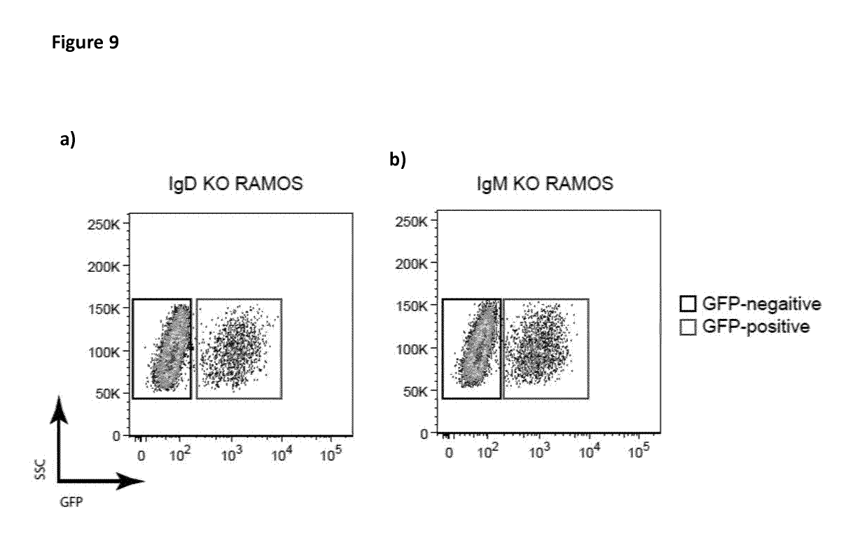

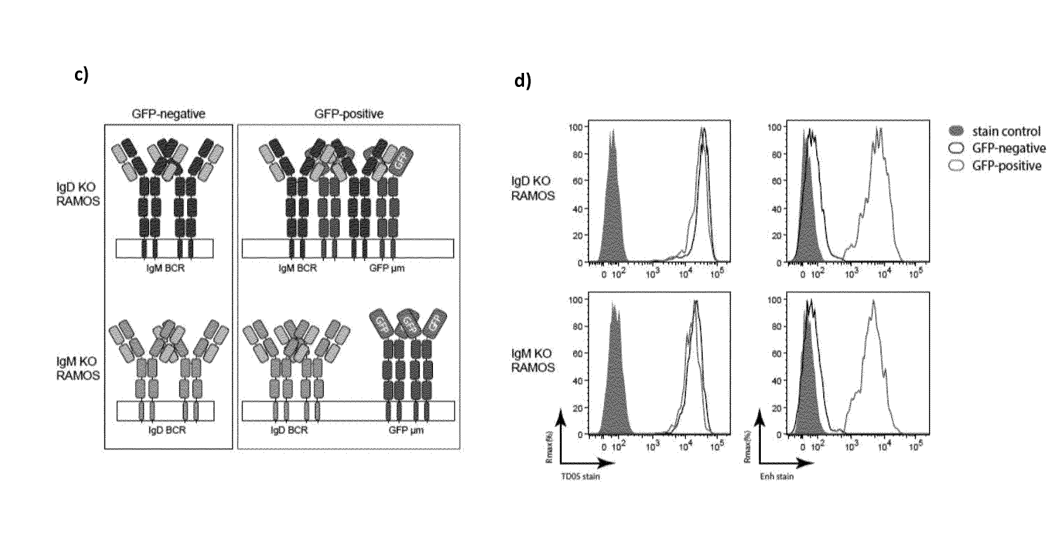

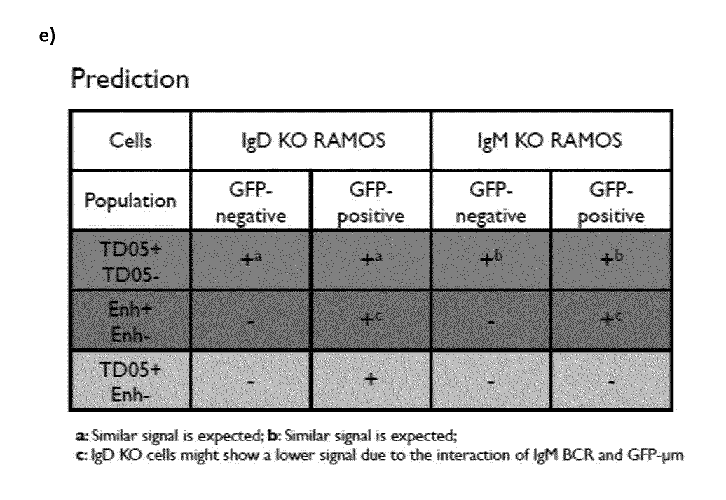

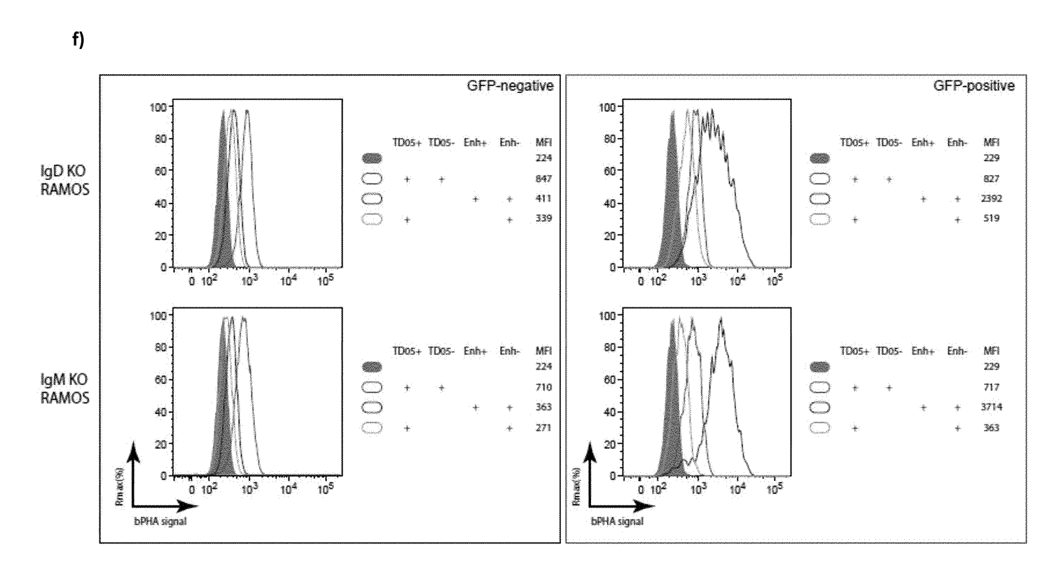

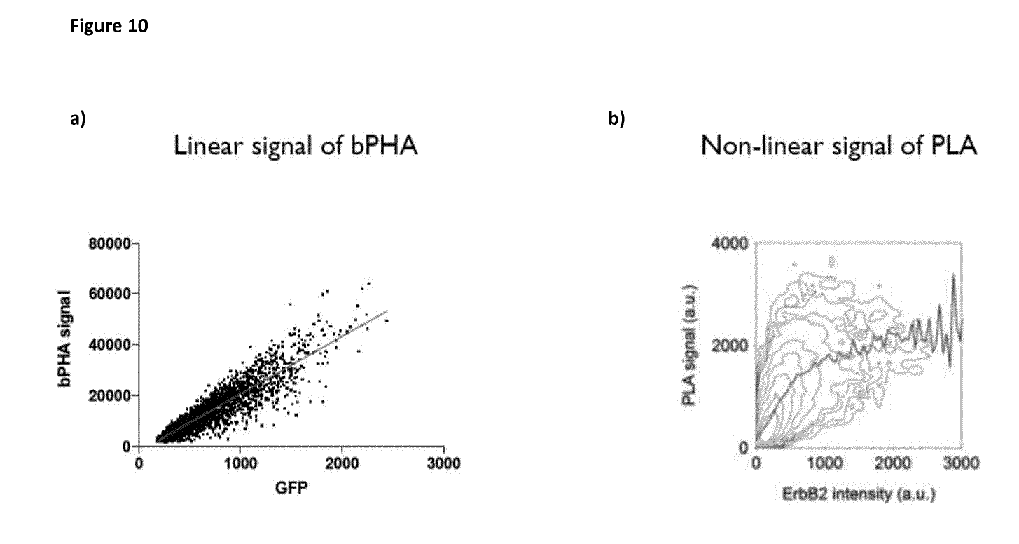

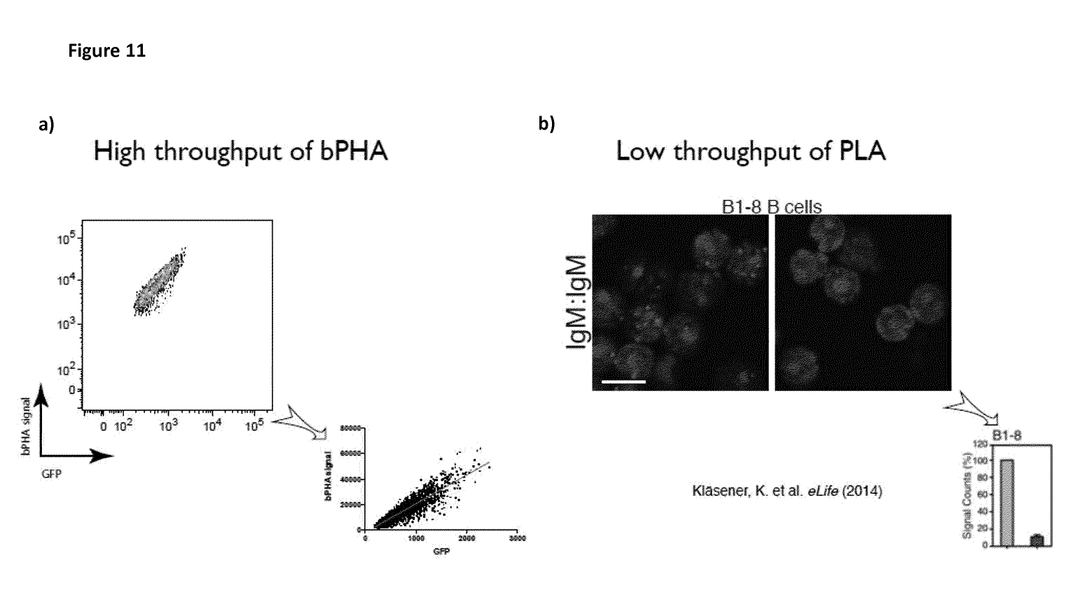

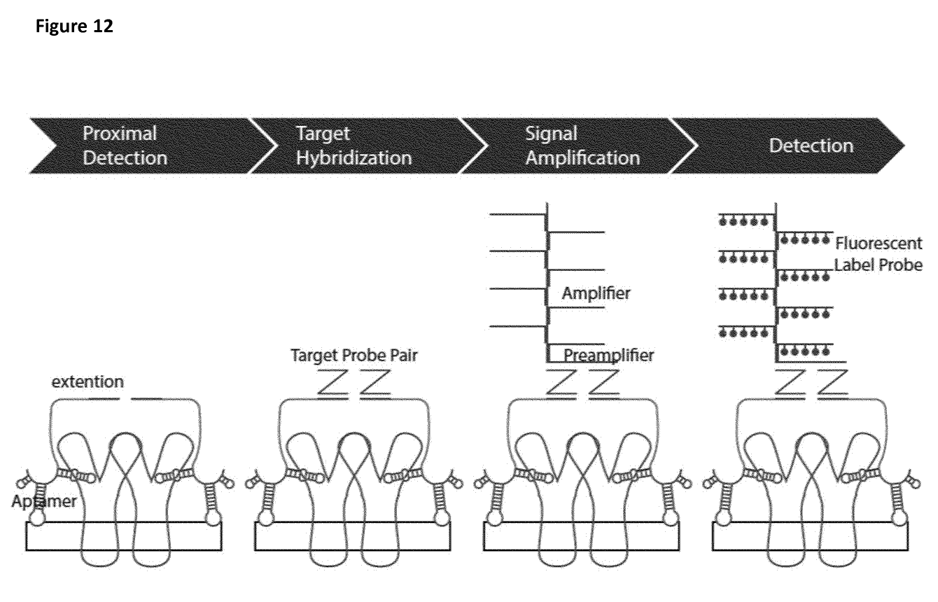

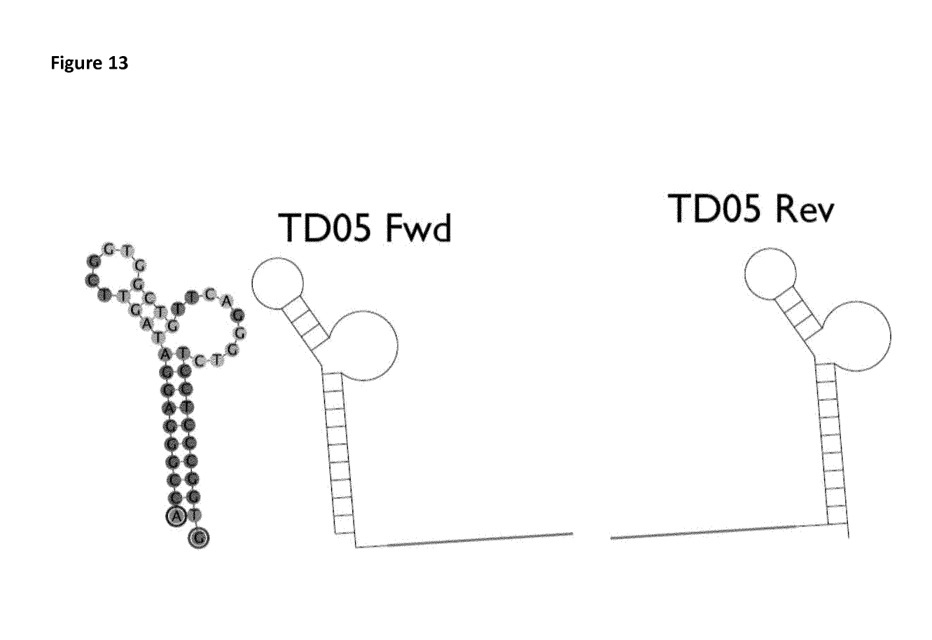

FIG. 1: Schematic representation of the design of a preferred protein-protein proximity assay of the invention. The different steps of the method are demonstrated. This figure shows a method using a Z-DNA Probe. The target biomolecules are two different proteins A and B. Two Target Binding Reagents are used, both protein binding reagents. Both Target Binding Reagents have an attached Oligo Extension comprising a sequence complementary to a Z-DNA Probe. bDNA technology is used for the detection.
FIG. 2: Schematic representation of the design of a preferred protein-nucleic acid proximity assay of the invention. The target biomolecules are a protein and a nucleic acid target. Two different Target Binding Reagents are used, one protein binding reagent and one oligonucleotide specific for the nucleic acid target. Both Target Binding Reagents have an attached Oligo Extension comprising a sequence complementary to a Z-DNA Probe. bDNA technology is used for the detection.
FIG. 3: Example of Oligo Extension couplings to Target Binding Reagents via chemical crosslinking. As a Target Binding Reagent an antibody, a Fab fragment, a nanobody, or a scFv can be used. Sulfo-SMCC (sulfosuccinimidyl 4-(N-maleimidomethyl)cyclohexane-1-carboxylate) and a thio modified oligonucleotide are used for the crosslinking.
FIG. 4: Example of Oligo Extension couplings to Target Binding Reagents using sortase transpeptidase. This figure shows the specific labeling of a target specific scFv or nanobody with only one oligonucleotide using sortase transpeptidase (1:1 molar ratio).
FIG. 5: Example of Oligo Extension couplings to Target Binding Reagents using aptamers. A 1:1 Oligo Extension/Target Binding Reagent ratio using aptamers is achieved.
FIG. 6: Protein-protein proximity proof of concept: Detection of BCR oligomers on the Ramos cell surface using aptamers as Target Binding Reagents. FIG. 6 a) shows a schematic representation of the detection. FIG. 6 b) shows that the resting state BCR oligomer can be detected by a preferred method of the invention using aptamer as target binding reagents. Control without the Z-DNA Probes produces negative results.
FIG. 7: Protein-protein proximity proof of concept: Detection of the membrane associated immunoglobulin heavy chain dimers on the Ramos cell surface using nanobodies as target binding reagents. FIG. 7 a) shows a schematic representation of the detection. FIG. 7 b) shows that the dimerization of the membrane associated immunoglobulin dimer can be detected by a preferred method of the invention using nanobodies as Target Binding Reagents. Controls missing the Z-DNA Probes or one of the Target Binding Reagents produce negative results.
FIG. 8: Protein-protein proximity proof of concept: Detection of cytokine receptor homodimerization on the cell surface using antibodies as Target Binding Reagents. FIG. 8 a) shows that the homodimerization of TSLPR on the J558L cell surface is only detected in TSLPR expressing cells by a preferred method of the invention. FIG. 8 b) shows that the homodimerization of IL-7Ra on the J558L cell surface is only detected in the II-7Ra expressing cells by a preferred method of the invention.
FIG. 9: Results of bPHA experiments. FIG. 9 a) shows the FACS results for the transduced IgD KO Ramos cells and the gating strategy. The GFP negative cells express only the IgM BCR while the GFP positive population expresses the GFP-μm as well. FIG. 9 b) shows the FACS plot for the transduced IgM KO Ramos cells including the gating strategy. The GFP negative cells express only the IgD BCR while the GFP positive population expresses the GFP-μm as well.
   FIGS. 9 c)-f) show that bPHA Target Binding Reagents are specific.
   FIG. 9 c) shows a schematic representation of the surface BCR organization in the transduced (GFP positive) and non-transduced (GFP negative) IgD KO or IgM KO Ramos cells. The GFP negative IgD KO cells express only the IgM-BCR which should form BCR oligomers while the GFP positive population expresses the GFP-μm as well that incorporates into the IgM-BCR oligomers. The GFP negative IgM KO cells express only the IgD-BCR which forms BCR oligomers while the GFP positive population would express the GFP-μm as well and it could not incorporate into the IgD-BCR oligomers.
   FIG. 9 d) shows that the bPHA Target Binding Reagents are specific. GFP-μm expression in IgD KO and IgM KO Ramos cells are equal and the expression of GFP-μm did not alter the surface expression of both IgM-BCR and IgD-BCR in the IgD KO and IgM KO Ramos cells. Fluorescent labeled TD05 was able to detect similar expression of IgD-BCR and IgM-BCR in both the transduced and non-transduced IgD KO and IgM KO cells suggesting that the expression of GFP-μm did not alter the surface expression of both IgM-BCR and IgD-BCR in the IgD KO and IgM KO Ramos cells. Fluorescent labeled Enh (GFP-specific nanobody called enhancer) could detect a similar expression of GFP-μm only in the transduced cells.
   FIG. 9 e) explains the expected bPHA results using different pairs of the Target Binding Reagents based on the scheme of FIG. 9 c). Basically, the TD05+/TD05− pair allows the detection of Ramos BCR (IgM-BCR in IgD KO and IgD-BCR in IgM KO) oligomerization. Enh+/Enh− pair allows the detection of GFP-μm surface expression and the proximity of the two arms of the GFP-μm. The TD05+/Enh− pair allows the detection of the proximity between GFP-μm and the BCR.
   FIG. 9 f) shows that the bPHA results are as expected. The GFP-μm/BCR proximity can be seen in the transduced IgD KO cells. The signal is higher in the IgD KO cells then in the transduced IgM KO cells.
FIG. 10: FIG. 10 shows a comparison of linearity of a preferred method of the invention and a state of the art proximity ligation assay (PLA). FIG. 10 a) shows the linear signal of a bPHA method of the invention whereas FIG. 10 b) shows the non-linear signal of a PLA assay.
FIG. 11: FIG. 11 shows a comparison of data collection and analysis speed of a preferred method of the invention and a state of the art proximity ligation assay (PLA). FIG. 11 a) shows that the bPHA signal is measured for 2500 cells by FACS in only 24 sec. The raw data are exported for fitting and the fitting can be done within seconds. FIG. 11 b) shows that PLA signal is measured by confocal microscopy. The collection of images with 100-200 cells typically amounts to 1-2 hrs. The images are then processed by ImageJ, the PLA signals counted by the Blobfinder software, and the final counts plotted as bar graph, which normally requires 2-3 hours.
FIG. 12: Schematic representation of the design of a preferred protein-protein proximity assay of the invention using aptamers as Target Binding Reagents. Two Target Binding Reagents are used, both aptamers. Both Target Binding Reagents have an attached Oligo Extension comprising a sequence complementary to a Z-DNA Probe. bDNA technology is used for the detection.
FIG. 13: FIG. 13 shows the design of preferred aptamer Target Binding Reagents with attached Oligo Extensions.

### Examples

The invention is further described by the following examples. These are not intended to limit the scope of the invention. The experimental examples relate to

### 1. Protein-Protein Proximity Assay Using an Aptamer

Proof of concept was demonstrated with a detection of BCR oligomers on the Ramos cell surface. Therefore, the target proteins were BCR on the Ramos cell surface. As the Target Binding Reagent a synthesized TD05 aptamer was used. Different Oligo Extensions, each with a complementary region to a Z-DNA Probe, were used in this example.

FIG. 6 a) shows a schematic representation of this detection. FIG. 6 b) shows that the resting state BCR oligomer can be detected by a preferred method of the invention using aptamer as Target Binding Reagents. Control without the Z-DNA Probes produces negative results. As a Ramos BCR-specific TD05 aptamer the following sequence was used.

SEQ ID NO 7:
ACCGGGAGGATAGTTCGGTGGCTGTTCAGGGTCTCCTCCCGGTG

TD05+, TD05− and TD05+− were generated by synthesis. TD05+− alone can detect the BCR expressed on the Ramos cells. “+” and “−” refer to different Oligo Extensions.

TD05+ and TD05− pairs are needed to detect the BCR (oligomers).

### 2. Protein-Protein Proximity Assay Using a Nanobody

GFP fused to mIgM HC (GFP-μm) was used as target biomolecules. The target binding reagent was the “enhancer” (Enh) GFP-specific nanobody produced in E. coli.

Enh+, Enh− were generated by sortagging. FIG. 7 shows in a) a structure of the hybridized complex. FIG. 7 b) shows the bPHA can be used to detect the proximity of the two arms of the BCR.

### 3. Protein-Protein Proximity Assay Using an Antibody

Detection of cytokine receptor homodimerization on the cell surface

TSLPR or IL-7Ra expressed on the cells surface were used as target proteins. Target Binding Reagents are anti-TSLPR antibody and anti-IL-7Ra antibody (both are commercially available: Anti-TSLPR: R&D, AF546, anti-II7ra(CD127): eBioscience, 14-1271-82). Oligo Extension coupled antibody probes were prepared using sulfo-SMCC and thio modified oligonucleotides.

FIG. 8 a) shows that the homodimerization of TSLPR on the J558L cell surface is only detected in the TSLPR expressing cells by bPHA. FIG. 8 b) shows that the homodimerization of IL-7Ra on the J558L cell surface is only detected in the IL-7Ra expressing cells by bPHA.

### 4. Multiplexing

To confirm the class-specific oligomerization of the BCR, multiplexing bPHA experiments were conducted.

bPHA experimental systems:
IgD KO Ramos cells and IgM KO Ramos cells
Transduction with GFP-μm results in a mixed GFP positive and GFP negative population

The results of these experiments proof that bPHA Target Binding Reagents are specific. Therefore, bPHA faithfully detects the oligomerization of IgM and IgD and works in mixed cell populations.

The results are shown in FIG. 9.

### 5. Linearity

Linearity comparison of bPHA and the state-of-the-art proximity ligation assay (PLA) were performed.

The results are shown in FIG. 10.

### 6. Data Collection and Speed

Data collection and analysis speed comparison of bPHA and PLA were performed.

The results are shown in FIG. 11. bPHA of the invention:

Collection of 2500 cells by FACS: 24 seconds

Data analysis on laptops: 5 seconds

PLA (state-of-the-art):

Collection of 100-200 cells by microscopy: 1 to 2 hours

Data analysis on desktop computers: 2 to 3 hours

1. Method for detecting the proximity of at least two target biomolecules comprising:
Providing at least two target biomolecules,
providing at least two Target Binding Reagents, each binding to at least one of said target biomolecules,
wherein at least one designed Oligo Extension, comprising a linker and a complementary sequence to a Z-DNA Probe or a complementary sequence to a Pre-amplifier, is attached to each Target Binding Reagent,
binding of at least two Target Binding Reagents with different Oligo Extensions to the at least two target biomolecules and
hybridization of two Z-DNA Probes, that are in vicinity, to Pre-amplifiers, or hybridization of the sequence complementary to a Pre-amplifier to Pre-amplifiers,
forming a bDNA structure, wherein said bDNA structure comprises Pre-amplifiers and Amplifiers
hybridization of Label Probes and
detection.

2. Method according to claim 1, wherein the Target Binding Reagent is a biomolecule, preferred selected from the group comprising nucleic acid sequences, aptamers, antibodies, Fab, nanobodies and scFv.

3. Method according to claim 1 or 2, wherein said two Z-DNA Probes bind to the complementary sequences of the Oligo Extensions.

4. Method according to at least one of the preceding claims, wherein the molar ratio between Target Binding Reagent and Oligo Extension is 1:1.

5. Method according to at least one of the preceding claims, wherein the method is a high-throughput method.

6. Method according to at least one of the preceding claims, wherein the detection is a fluorescent or an enzymatic detection.

7. Method according to at least one of the preceding claims, wherein the method is a multiplex assay.

8. Method according to at least one of the preceding claims, wherein the target biomolecules are proteins or nucleic acids.

9. Method according to at least one of the preceding claims, wherein the method is a protein-protein and/or a protein-nucleic acid proximity assay.

10. Method according to at least one of the preceding claims, wherein the Label Probes are fluorescently, biotin- or enzyme-labeled probes.

11. Method according to at least one of the preceding claims, wherein the method is linear over at least two logarithmic scales.

12. Method according to at least one of the preceding claims, wherein the designed Oligo Extension is coupled to a Target Binding Reagent via
(i) Chemical crosslinking
(ii) Labeling Target Binding Reagent with a designed Oligo Extension using a sortase transpeptidase,
(iii) Nucleic acid synthesis.

13. Kit for use in a method according to at least one of the preceding claims, comprising Target Binding Reagents with attached designed Oligo Extensions and Pre-amplifiers, Amplifiers and Label Probes.

14. Kit according to the preceding claim further comprising Z-DNA Probes.

15. Use of a method or a kit according to at least one of the preceding claims in medical diagnostics.
